(19) United States
(12) Patent Application Publication
(10) Pub. No.: US 2021/0197197 A1
(43) Pub. Date: Jul. 1, 2021

(54) LATERALLY-DISPLACED MICROPOST ARRAY CHIP AND USE THEREOF

(71) Applicant: ZIGZAG BIOTECHNOLOGY LTD., Shenzhen (CN)

(72) Inventor: Zongbin LIU, Shenzhen, Guangdong (CN)

(21) Appl. No.: 17/058,497

(22) PCT Filed: May 27, 2019
(86) PCT No.: PCT/CN2019/088535

§ 371 Date: Nov. 24, 2020

(30) Foreign Application Priority Data
Jun. 6, 2018 (CN) 201810573629.3

(51) Int. Cl. B01L 3/00

(52) U.S. Cl. CPC B01L 3/502761; B01L 2200/0652; B01L 2200/027

(57) ABSTRACT

The disclosure provides a laterally-displaced micropost array chip and the use thereof. The laterally-displaced micropost array chip includes laterally-displaced micro-pillar arrays arranged in columns or in rows. Micropost units in each subsequent column or subsequent row are displaced relative to a previous column or previous row according to a certain angle. Each of the micropost units is internally provided with one or more channels. In the one or more channels, the opening direction of at least one channel is different from the displacement directions of the laterally-displaced micropost arrays. The laterally-displaced micropost array chip of the invention more accurately separates particles of various sizes in fluids, has the function of filtering particles of small sizes, thereby enabling particles of large sizes to have an enrichment effect before entering the next array, reduces the critical separation sizes of micropost arrays, and is higher in throughput and larger in separation volume.

### CROSS REFERENCE TO RELATED APPLICATIONS

This application is the national phase entry of international Application No. PCT/CN2019/088535 filed on May 27, 2019 for which priority is claimed under 35 U.S.C. § 120; and this application claims priority of Application No. 201810573629.3 filed in CHINA on 6 Jun. 2018 under 35 U.S.C. § 119, the entire contents of all of which are hereby incorporated by reference.

### FIELD OF TECHNOLOGY

The present disclosure relates to a technical field of sorting cells, and specifically in particular to a laterally-displaced micropost array chip and use therefore.

### BACKGROUND OF THE INVENTION

It is a basic analysis method to sort substances or particles according to sizes of substances or particles in technical fields of biology, medicine, chemistry and industry, commonly used methods including filtration, chromatograph, inertia force, vortex and laterally-displaced micropost arrays and so on. The technology of laterally-displaced micropost arrays having features of micropost barrier arrays arranged in columns or in rows and a subsequent column or row displaced at a certain angle relative to a previous column is widely used because of its size-based precise isolation, and every micropost array has a specific substance critical sorting size (diameter) according to its size and angle arrangement of the micropost array. Large particles greater than the critical diameter move in a direction of displacement angle after being collided with microposts, while particles smaller than critical diameter keep an original flow direction, then large particles and small particles are spatially separated. It has been reported that some microfluidic chips are designed with lateral displacement micropost arrays to separate erythrocytes, leukocytes, circulating tumor cells and circulating fetal nucleated red blood cells etc.

The cross-section shapes of microposts in present lateral displacement arrays comprise shapes of circle, triangle, rectangle, rhombus, or H-shaped and so on. In order to get a greater critical sorting size, a smaller displacement angle would be required, because of the greater displacement angle of micropost arrays, the smaller sorting sizes (diameter of particle). If a length of a sorting channel is fixed, a smaller displacement angle would result in a narrower sorting chamber which would lead to a clog easily and lower throughput, so this kind of chips or equipments can't separate big volume samples, which may limit their application.

### SUMMARY OF THE INVENTION

The present disclosure provides a lateral displacement micropost array chip and the use method thereof, every micropost unit in the chip has one or more channels. When fluid consisting of particles with various size passes through micropost units, small particles would pass through small channels inside of units, but big ones would not and keep an original flow direction. The micropost structure has an enrichment effect for big particles by filtrating small particles before big particles flow into the subsequent arrays. Under the same displacement angles of micropost arrays, the critical sorting size of the micropost arrays can be decreased; and under the same micropost size, a same critical sorting size can be obtained by adopting a large array displacement angle, and a larger array displacement angle can produce a higher throughput for separating big-volume samples.

The first purpose of present disclosure is to provide a lateral displacement micropost array chip, and each micropost unit consists of one or more channels.

The body of the lateral displacement micropost array chip provided by the present disclosure is well-known in traditional art.

In an embodiment of the present disclosure, each of the micropost units can be provided with 1 to 3, 1, 2, or 3 channels.

In the present disclosure, particles both smaller than the critical sorting size of lateral displacement micropost arrays and size of channel cross sections can pass through channels; particles greater than the critical sorting size of lateral displacement micropost arrays can't pass through channels and keep an flow direction of lateral displacement.

The critical sorting size of a micropost structure is the size that particles greater than it can be collected completely in an outlet for target particles, and particles smaller than it can be collected completely in a waste liquid outlet.

The body of the laterally-displaced micropost array chip includes micropost barrier arrays (That is, lateral displacement micropost arrays) which arranged in columns or in rows, and every subsequent column or row is displaced at a certain angle relative to the previous column or row.

The smallest size of channel cross sections in a laterally-displaced micropost array chip could be nano-level or micro-level. In the embodiments of the present disclosure, the smallest size of channel cross sections is smaller than the critical sorting size of lateral displacement micropost arrays.

Inside a laterally-displaced micropost array chip, one opening direction of at least one channel of one or more channels is different from the displacement direction of lateral displacement micropost arrays. In one embodiment of the present disclosure, multi-channels share one opening.

Shapes of channel cross sections of a chip could be any regular or irregular shapes. In an embodiment of the present disclosure, the shape is L-shaped. Shapes of micropost cross sections could be any regular or irregular shapes. In an embodiment of the present disclosure, shapes could be triangular, rectangular, L-shaped or other irregular shapes as illustrated in figures.

Each micropost unit of the aforementioned chip is composed of two or more independent microposts, and gaps between microposts form channels.

The size of micropost units of the chip could be nano-level or micro-level.

Above laterally-displaced micropost array chip is made of one or more of glass, silicon and a polymer; and the polymer is at least one of polymethyl methacrylate, bisphenol A polycarbonate, polycarbonates of 2,2-bis(4-hydroxyphenyl)propane, polystyrene, polyethylene, silicon resin, polyvinyl acetate, polypropylene, polyvinyl chloride, polyether ether ketone, polyethylene glycol terephthalate, cycloolefin polymer and cycloolefin copolymer. The cycloolefin of cycloolefin polymer and cycloolefin copolymer is selected from one or more of cyclopropene, cyclobutene, cyclopentene, cyclohexene, 1,3-Cyclobutadiene, cyclopentadiene and cyclohexadiene.

In a laterally displaced micropost array chip, except the design of micropost units in the chip, the structure can adopt any being-known laterally-displaced micropost array chip designs. In an embodiment of the present disclosure, a chip comprises a substrate and/or a cover hermetically matched with the substrate; the substrate or the cover is provided with lateral displacement micropost arrays; one end of the chip is provided with a sample inlet to serve as a liquid sample inlet and/or buffer solution inlet, and the other end of the chip is provided with a waste liquid outlet used for collecting particles smaller than the critical sorting size and a target particles outlet used for collecting enriched particles greater than the critical sorting size. Lateral displacement micropost arrays in substrates or covers are arranged unilaterally or bilaterally.

The second purpose of present disclosure is to provide a method using the laterally-displaced micropost array chip above for sorting a liquid sample composed of various size particles, comprising following steps: flowing a liquid sample consisting of various size particles through lateral displacement micropost arrays. Particles greater than the critical sorting size flow along the displacement angle direction of the lateral displacement arrays to be collected in a target particles outlet, and particles both smaller than the critical sorting size and the channel smallest size keep an original direction to be collected in a waste liquid outlet. Particles of various sizes are spatially separated.

The samples in the method above could be any one of the followings:
(1) a Circulating Tumor Cell (CTCs) in a peripheral blood sample;
(2) a tumor cell in a pleural effusion, peritoneal effusion, lymph fluid, urine or bone marrow sample;
(3) a nucleated erythrocyte in a peripheral blood or umbilical cord blood sample;
(4) a circulating endothelial cell in the peripheral blood sample;
(5) a leukocyte, a T cell, a B cell, a lymphocyte, a monocyte, a granulocyte, a natural killer cell, a dendritic cell, a macrophage or a hematopoietic stem cell in a peripheral blood, umbilical cord blood, pleural effusion, peritoneal effusion, urine, cerebrospinal fluid or bone marrow sample;
(6) an erythrocyte or a platelet in a peripheral blood, umbilical cord blood, pleural effusion, peritoneal effusion, urine or bone marrow sample;
(7) a bacterium or a virus in a peripheral blood, pleural effusion, peritoneal effusion, urine, saliva, plasma, serum, cerebrospinal fluid, seminal fluid, prostatic fluid or vaginal secretion sample; and
(8) a sperm in a seminal fluid sample.

The third purpose of the present disclosure is to provide the chip's application for any one of the followings:

(1) a Circulating Tumor Cell (CTCs) in a peripheral blood sample;
(2) a tumor cell in a pleural effusion, peritoneal effusion, lymph fluid, urine or bone marrow sample;
(3) a nucleated erythrocyte in a peripheral blood or umbilical cord blood sample;
(4) a circulating endothelial cell in the peripheral blood sample;
(5) a leukocyte, a T cell, a B cell, a lymphocyte, a monocyte, a granulocyte, a natural killer cell, a dendritic cell, a macrophage or a hematopoietic stem cell in a peripheral blood, umbilical cord blood, pleural effusion, peritoneal effusion, urine, cerebrospinal fluid or bone marrow sample;
(6) an erythrocyte or a platelet in a peripheral blood, umbilical cord blood, pleural effusion, peritoneal effusion, urine or bone marrow sample;
(7) a bacterium or a virus in a peripheral blood, pleural effusion, peritoneal effusion, urine, saliva, plasma, serum, cerebrospinal fluid, seminal fluid, prostatic fluid or vaginal secretion sample; and
(8) a sperm in a seminal fluid sample.

### BRIEF DESCRIPTION OF THE DRAWINGS

FIGS. 1 and 2 are structure schematic diagrams of a laterally-displaced micropost array chip of the present disclosure.

FIG. 3 is a schematic diagram of the cross section of the composite micropost 1.

FIG. 4 is a schematic diagram of the cross section of the composite micropost 2.

FIG. 5 is a schematic diagram of the cross section of the composite micropost 3.

FIG. 6 is a schematic diagram of the cross section of the composite micropost 4.

FIG. 7 is a schematic diagram of the cross section of the composite micropost 5.

FIG. 8 is a flow direction schematic diagram when a liquid flows through the composite micropost shown in FIG. 5.

FIG. 9 is a sorting schematic diagram for large, medium and small particles when a sample fluid consisting of various size particles pass through the structure shown in FIG. 5.

FIG. 10 is a throughput comparison schematic diagram for the composite micropost array structures with shapes of a circle, a triangle and the structure shown in FIG. 5.

FIG. 11 is a structure schematic diagram of the laterally-displaced micropost array chip of the present disclosure.

### REFERENCE NUMBERS IN DRAWINGS SHOWS

Substrate 1, cover 2, micropost unit 3, sample inlet 4, target particles outlet 5, waste liquid outlet 6, buffer solution inlet 7.

### DESCRIPTION OF THE EMBODIMENTS

Unless particularly stated, the experimental methods used in the following embodiments are common methods.

Unless particularly stated, the reagents, materials etc. used in the following embodiments could be acquired commercially.

Following embodiments are used for illustrating present disclosure rather than limiting the scope of present disclosure.

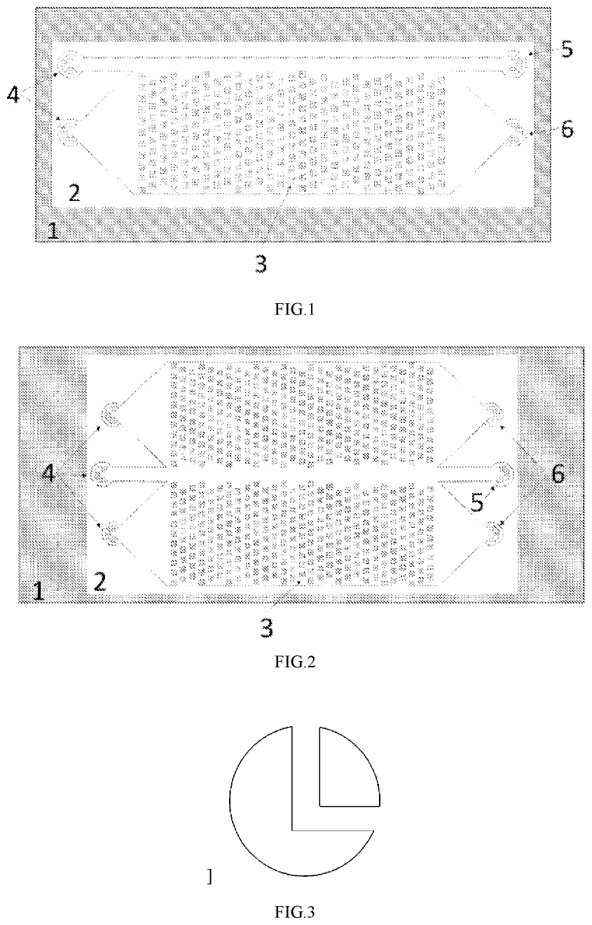

As shown in FIG. 1 or 2, a chip provided by the present disclosure comprises a substrate 1 and/or a cover 2 hermetically matched with the substrate 1; and both of them are made of one or more of glass, silicon and a polymer. the polymer is at least one of polymethyl methacrylate, bisphenol A polycarbonate, polycarbonates of 2,2-bis(4-hydroxyphenyl)propane, polystyrene, polyethylene, silicon resin, polyvinyl acetate, polypropylene, polyvinyl chloride, polyether ether ketone, polyethylene glycol terephthalate, cycloolefin polymer and cycloolefin copolymer. The cycloolefin of cycloolefin polymer and cycloolefin copolymer is selected from one or more of cyclopropene, cyclobutene, cyclopentene, cyclohexene, 1,3-Cyclobutadiene, cyclopentadiene and cyclohexadiene.

Lateral displacement micropost arrays inside of a substrate 1 or a cover 2 are arranged unilaterally (FIG. 1) or bilaterally (FIG. 2). A chip provided by the present disclosure is provided with lateral displacement micropost arrays which arranged in columns or in rows, and each subsequent column or row is displaced at a certain angle relative to the previous columns or rows. The size of a micropost unit inside of a chip could be nano-level or micro-level, each micropost unit 3 with one or more circular, triangular, rectangular or special-shaped structures in cross section is composed of two or more independent microposts, and gaps between microposts form one or more channels. One opening direction of at least one channel is different from the displacement direction of the lateral displacement micropost arrays, to be specific, at least one L-shaped channel cross section included, with its smallest size (width or height) of the channel cross section smaller than the critical sorting size of a chip which allowing particles smaller than the channel cross section to pass through the channel. One end of the chip is provided with a sample inlet 4 to serve as a liquid sample inlet and/or buffer solution inlet, and the other end of the chip is provided with a waste liquid outlet 6 used for collecting particles smaller than the critical sorting size and a target particles outlet 5 used for collecting particles greater than the critical sorting size.

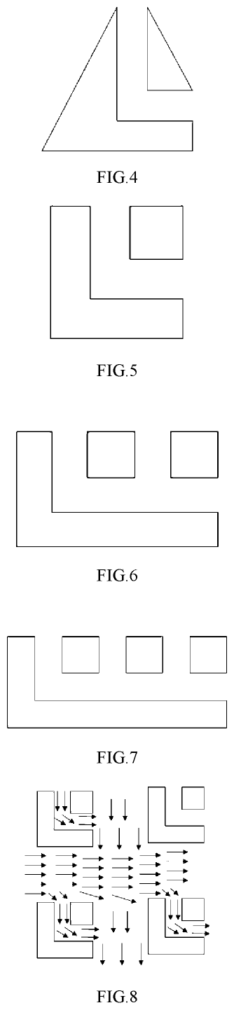

To be specific, each micropost unit is composed of two independent microposts so that a gap between the two microposts forms a channel as shown in FIG. 3 to FIG. 5, and shapes of cross sections of two microposts could be circular, trianglar, rectanglar or special-shaped structures.

To be specific, as shown in FIG. 6, each micropost unit is composed of three independent microposts, the cross sections of three microposts are respectively L-shaped, rectanglar and rectanglar. Gaps among microposts form two channels with L-shaped cross sections. Two channels with L-shaped cross sections share the same outlet.

To be specific, as shown in FIG. 7, each micropost unit is composed of four independent microposts, the cross sections of four microposts are respectively L-shaped, rectanglar, rectanglar, and rectanglar. Gaps between microposts form three channels with L-shaped cross sections. Three channels with L-shaped cross sections share a same outlet.

When using a chip, a sample fluid consisting of various size particles and/or a buffer solution without particles enter into a chip from sample inlets, as shown in FIG. 8 and FIG. 9, then flow through lateral displacement micropost arrays, in the flowing process, paths of particles greater than the critical sorting size show in FIG. 9 in full lines, that particles flow along the displacement angle of lateral displacement micropost arrays, and paths of particles smaller than the smallest size (width or height) of channel cross sections show in broken lines, that part of particles flow through channels, part of particles flow into longitudinal runners and then be enriched in transverse runners below and finally keep an original direction movement; particles the size between the critical sorting size and the smallest size (width or height) of the channel cross sections flow into longitudinal runners and then be enriched transverse runners below and finally keep an original direction movement. Particles with various sizes are spatially separated, that particles greater than the critical sorting size flow out from a target particles outlet, and particles smaller than the critical sorting size flow out from a waste liquid outlet. The micropost unit of the present disclosure is a kind of composite micropost having an enrichment effect for big particles by filtrating small particles before big particles flow into the subsequent arrays.

The chip provided by the present disclosure is used for sorting microparticles or nanoparticles of liquid samples, such as cells, bacteria, viruses etc. in biological samples, including but not limited to any one of below: (1) separating Circulating Tumor Cells (CTCs) in a peripheral blood sample; (2) separating tumor cells in a pleural effusion, peritoneal effusion, lymph fluid, urine or bone marrow sample; (3) separating nucleated erythrocytes in a peripheral blood or umbilical cord blood sample; (4) separating circulating endothelial cells in the peripheral blood sample; (5) separating leukocytes, T cells, B cells, lymphocytes, monocytes, natural killer cells, dendritic cells, macrophages or hematopoietic stem cells in a peripheral blood, umbilical cord blood, pleural effusion, peritoneal effusion, urine, cerebrospinal fluid or bone marrow sample; (6) separating erythrocytes or platelets in a peripheral blood, umbilical cord blood, pleural effusion, peritoneal effusion, urine or bone marrow sample; (7) separating bacterium or virus in a peripheral blood, pleural effusion, peritoneal effusion, urine, saliva, plasma, serum, cerebrospinal fluid, seminal fluid, prostatic fluid or vaginal secretion sample; and (8) separating sperms in a seminal fluid sample.

### Embodiment 1

Taking a micropost unit structure of the lateral displacement micropost array chip illustrated in FIG. 5 for example, to evaluate the sorting effect of the chip provided by the present disclosure, the substrate is made of inorganic glass and the cover is made of polydimethylsiloxane.

I. Critical Sorting Sizes of Different Micropost Structures Having the Same Array Sizes and Displacement Angles

In order to compare the influences that different micropost structures to the critical sorting sizes, inlets and outlets of chips shown in FIG. 1 are adopted, and the micropost array structures in chips are respectively circular microcolumns, triangular microcolumns and the composite microcolumns shown in FIG. 5. Circular microcolumns have diameters of 10 μm, row gaps of 10 μm, micropost gaps of 10 μm, and array lateral displacement angles of 6 degree; triangular microcolumns have base lines of 10 μm long, heights of 10 μm, row gaps of 10 μm, micropost gaps of 10 μm, and array lateral displacement angles of 6 degree; composite microcolumns have both lengths and widths of 10 μm, row gaps of 10 μm, micropost gaps of 10 μm, and array lateral displacement angles of 6 degree; width of small channels of composite microcolumns is 2 μm, and both length and width of small rectangle microcolumns are 4 μm. The height of all microcolumns above is 10 μm.

Three critical sorting sizes for above microcolumn structures having the same array sizes and same displacement angles are obtained by following steps: Passing PBS buffer solutions (pH 7.2 to 7.4, NaCl 137 mmol/L, KCl 2.7 mmol/L, Na₂HPO₄10 mmol/L, KH₂PO₄ 2 mmol/L) and PBS buffer solutions containing the fixed-size polystyrene microparticles through top inlets and bottom inlets of above three chips respectively with volume ratios of 1:1-1:5. The size of the fixed-size microparticles are respectively 2 μm, 3 μm, 4 μm and 5 μm. Both of solutions flow through lateral displacement micropost arrays together at 3-5 mm/s. In the flowing process, microparticles with various sizes are sorted, a waste liquid outlet 6 and a target particles outlet 5 are used respectively to collect the waste liquid and the particles enrichment liquid. Then observe the sizes of the enrichment liquid and waste liquid under a microscope. In a micropost structure, if particles greater than a certain size can be collected in a target particles outlet 5 and particles smaller than the size can't be completely collected in a waste liquid outlet 6, then the size is namely the critical sorting size of the microcolumn. Table 1 gives a critical sorting size statistical result of above three microcolumn structures, which shows that composite microcolumns provide a smallest critical sorting size under the same condition of array sizes and displacement angles, it can be concluded that composite microposts provided by the present disclosure can reduce critical sorting sizes significantly.

TABLE 1
Comparison of the critical sorting sizes of circular microcolumns,
triangular microcolumns and composite microcolumns

| | Circular | Triangular | Composite microcolumns |
|---|---|---|---|
| | microcolumns | microcolumns | having both of lengths and |
| | having | having base | widths of 10 μm, row gaps |
| | diameters of | lines long of 10 | of 10 μm, micropost gaps |
| | 10 μm, row | μm, heights of | of 10 μm, and array lateral |
| | gaps of 10 | 10 μm, row | displacement angles of 6 |
| | μm, micro- | gaps of 10 μm, | degree; width of small |
| | post gaps of | micropost gaps | channels in composite |
| | 10 μm, and | of 10 μm, and | microposts is 2 μm, and |
| | array lateral | array lateral | both lengths and widths |
| | displacement | displacement | of small rectangle micro- |
| | angles | angles | posts in composite |
| | of 6 degree | of 6 degree | microposts are 4 μm. |
| CRITICAL | ~5 μm | ~4 μm | ~3 μm |
| SORTING | | | |
| SIZE | | | |
| (Arrays | | | |
| lateral | | | |
| displacement | | | |
| is 6 degree) | | | |

II. Displacement Angles of Different Micropost Structures Having the Same Array Sizes and Critical Sorting Sizes

In order to compare the influences that different micropost array lateral displacement angles to the sorting size, inlets and outlets of chips shown in FIG. 1 are adopted, and micropost array structures in chips are respectively circular microcolumns, triangular microcolumns and the composite microcolumns shown in FIG. 5. Circular microcolumns have diameters of 10 μm, row gaps of 10 μm, micropost gaps of 10 μm, and respective array lateral displacement angles of 3, 3.5, 4, 4.5, 5, 5.5, 6, 6.5, 7, 7.5, 8, 8.5, 9, 9.5 and 10 degree; triangular microcolumns have base lines of 10 μm long, heights of 10 μm, row gaps of 10 μm, micropost gaps of 10 μm, and respective array lateral displacement angles of 3, 3.5, 4, 4.5, 5, 5.5, 6, 6.5, 7, 7.5, 8, 8.5, 9, 9.5 and 10 degree; composite microcolumns have both of lengths and widths of 10 μm, row gaps of 10 μm, micropost gaps of 10 μm, and respective array lateral displacement angles of 3, 3.5, 4, 4.5, 5, 5.5, 6, 6.5, 7, 7.5, 8, 8.5, 9, 9.5 and 10 degree; width of small channels in composite microposts is 2 μm, and length and width of small rectangle microposts in composite microposts are 4 μm. The height of all microcolumns above is 10 μm.

Displacement angles of above three different microcolumn structures having the same array sizes and critical sorting sizes are obtained by following steps: Passing PBS buffer solutions and PBS buffer solutions containing particles about 4 μm in diameter through top inlets and bottom inlets of above three chips having same array sizes and critical sorting sizes respectively at 3 to 5 mm/s and volume ratios of 1:1 to 1:5; waste liquid outlets 6 and target particles outlets 5 are used respectively to collect waste liquids and particle enrichment liquids, then observe sizes of enrichment liquids and waste liquids under a microscope. In a micropost structure, if displacement angles of arrays smaller than a certain value, particles about 4 μm in diameter can be collected completely in a waste liquid outlet 6, if greater than that of certain value, particles about 4 μm in diameter can't be collected completely in that waste liquid outlet 6, then the value is namely the critical sorting size for sorting particles about 4 μm in diameter. Table 2 gives an experimental result showing that a composite micropost array produces a greatest array displacement angle under the condition that a circular micropost array, a triangular micropost array and a composite micropost array have the same gaps to get the same critical sorting size (4 μm). It can be concluded that, compared with microcolumns with continual cross sections, if sizes of microcolumns are the same, the composite microcolumns provided by the present disclosure can get the same critical sorting size by adopting a greater array displacement angle.

TABLE 2
Comparison of the displacement angles of circular micropost arrays,
triangular micropost arrays and composite micropost arrays

| | Circular | Triangular | Composite micropost |
|---|---|---|---|
| | micropost | micropost | arrays having a row |
| | arrays having | arrays having | gap of 10 μm, a |
| | diameters of | a base line of | micropost gap of 10 μm; |
| | 10 μm, row | 10 μm long, a | width of small channels in |
| | gaps of 10 | height of 10 | composite microcolumns |
| | μm, | μm, a row gap | is 2 μm, and length and |
| | micropost | of 10 μm, | width of small rectangle |
| | gaps | a micropost | microposts in composite |
| | of 10 μm | gap of 10 μm | microcolumns are 4 μm |
| Lateral | ~4.5 degree | ~6 degree | ~9 degree |
| Displacement | | | |
| Angles(The | | | |
| critical | | | |
| sorting | | | |
| size is | | | |
| 4 μm) | | | |

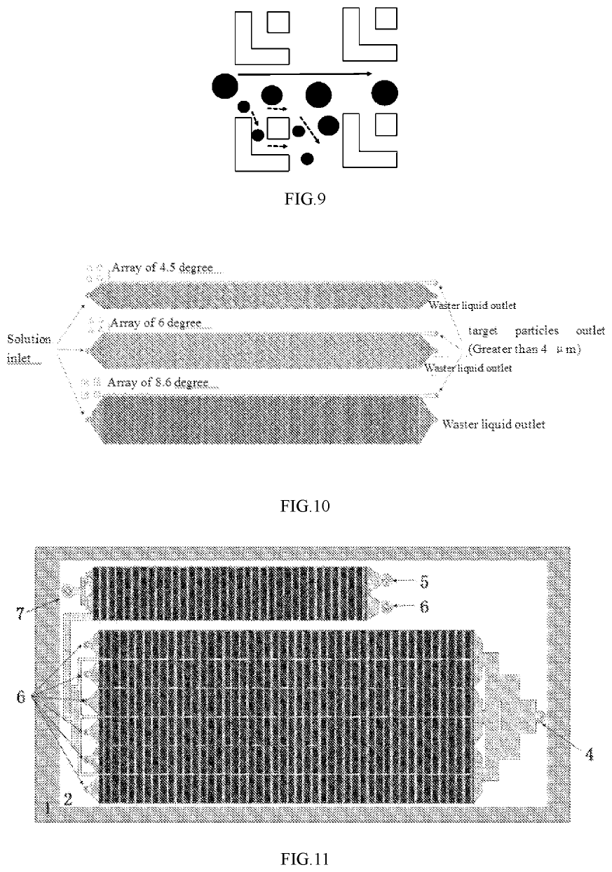

A greater displacement angle provides a higher throughput to separate bigger volume samples. As shown in FIG. 10, in order to enrich particles more than 4 μm in solution, the greatest angle provided by circular micropost arrays is 4.5 degree, provided by triangular micropost arrays is 6, and provided by composite micropost arrays is 9 degree. A chip with composite arrays has the biggest size in width, so the chip provided by the present disclosure provides a higher throughput at the same speed.

### Embodiment 2 Using a Composite Micropost Laterally-Displaced Chip to Sort Blood Cells

There are many cells in human blood, comprising erythrocytes, leukocytes, tumor cells and nucleated red blood cells etc. Erythrocytes have the smallest diameter about 3 to 5 μm; Leukocytes that diameters vary from 6 to 12 μm are subdivided into many subclasses, such as granulocytes, lymphocytes, monocytes and so on; Tumor cells that the diameter usually greater than 10 μm are often found in cancer patients' blood; Nucleated red blood cells that the diameter usually greater than 10 μm are often found in pregnant women blood. Chips with laterally-displaced composite micropost arrays can be used for sorting and enriching different cells in blood.

The inlet and outlet of a chip 1 is adopted to enrich tumor cells in human peripheral blood in this embodiment, and composite microcolumn structures in the chip 1 are shown in FIG. 5. Composite microcolumns have both lengths and widths of 15-70 row gaps of 20-70 μm, micropost gaps of 20-70 μm, and array lateral displacement angles of 2-12 degree; Width of small channels in composite microcolumns are 4-12 μm, and length and width of small rectangles in composite microcolumns are 3-30 um. Height of composite microcolumns above is 20-100 μm.

To be specific, a composite microcolumn has a length and a width of 50 μm, a row gap of 50 μm, a micropost gap of 50 μm, and an array lateral displacement angle of 3 degree; width of a small channel in a composite microcolumn is 10 μm, and a length and a width of a small rectangle micropost in a composite microcolumn is 10 μm, and the critical sorting size of the chip is about 10 μm.

Passing a PBS buffer solution (pH 7.2 to 7.4, NaCl 137 mmol/L, KCl 2.7 mmol/L, Na₂HPO₄ 10 mmol/L, KH₂PO₄ 2 mmol/L) and a blood sample containing tumor cells through a top inlet and a bottom inlet of a chip respectively with a volume ratio of 1:50-50:1. Both of the PBS buffer solution and blood sample flow through the chip together at 3 to 5 mm/s, in the flowing process, different size cells in blood are sorted, that tumor cells and big size leukocytes move in the direction of micropost array lateral displacement, erythrocytes and small size leukocytes move along the direction of liquid. A target particle (tumor cells) outlet 5 and a waste liquid outlet 6 are used respectively to collect tumor cell enrichment liquid and waste liquid after sorting.

The chip 1 and above method are adopted to sort tumor cells HepG2, and the concentration of tumor cells in a model sample is 124 cancer cells/ml. Table 3 gives a result of concentrations of tumor cells before and after sorting, showing that most of blood cells are filtrated, and tumor cells are enriched at an enrichment rate of 3.32×10⁴.

TABLE 3
Enrichment rates of tumor cells sorted by chip 1

| The ratio of HepG2 cells | The ratio of HepG2 cells | Enrichment rate |
|---|---|---|
| and blood cells(erythro- | and blood cells(small | of tumor cells |
| cytes and leukocytes) | amounts of residual | before and after |
| before sorting | leukocytes) after sorting | sorting |
| 124:1.02*10⁹ | 124:3.07*10⁴ | 3.32*10⁴ |

### Embodiment 3 Using a Composite Micropost Laterally-Displaced Chip to Sort Blood Cells

As there is only one inlet in the composite micropost laterally-displaced chip used for sorting blood cells in embodiment 2, the processing throughput is limited. In order to improve the processing throughput, a chip with symmetry composite microcolumns is used. Inlets and outlets of a chip shown in FIG. 2 is adopted in present embodiment, microcolumn structures inside of the chip is a kind of composite microcolumn shown in FIG. 5 with a length and a width of 15-70 μm, a row gap of 20-70 μm, a micropost gap of 20-70 μm, and an array displacement angle of 2-12 degree; width of small channels inside a composite micropost is 4-12 μm, length and width of small rectangle arrays inside a composite micropost is 3-30 μm, and height is 20-100 μm.

To be specific, a composite microcolumn has a length and a width of 50 μm, a row gap of 50 μm, a micropost gap of 50 μm, an array displacement angle of 3 degree; width of small channels inside a composite micropost is 10 μm, a length and a width of a small rectangle microcolumn inside a composite micropost is 10 μm, height is 50 μm, and the critical size of the chip is about 10 μm.

Passing a PBS buffer solution (pH 7.2 to 7.4, NaCl 137 mmol/L, KCl 2.7 mmol/L, Na₂HPO₄ 10 mmol/L, KH₂PO₄ 2 mmol/L) and a blood sample containing tumor cells respectively through three inlets with a volume ratio of 1:100-100:1, that PBS buffer solution flows through the middle inlet, blood samples flows through the top and bottom inlet. Both of PBS buffer solution and blood sample flow through lateral displacement micropost arrays together at 3-5 mm/s, in the flowing process, different size cells in blood are sorted, while tumor cells and big size leukocytes move in the direction of lateral displacement of micropost arrays, erythrocytes and small size leukocytes flow along the direction of liquid. A target particles (tumor cells) outlet 5 and a waste liquid outlet 6 are used respectively to collect tumor cells enrichment liquid and waste liquid after sorting.

A chip 2 and above method are adopted to sort hepatoma carcinoma cells HepG2, and the concentration of tumor cells in a model sample about 107 cancer cells per milliliter. Table 4 gives concentrations of tumor cells before and after sorting, showing that most of blood cells are filtrated, and tumor cells are enriched at an enrichment rate of 2.92×10⁴. The enrichment rates of tumor cells sorted by chip 1 and chip 2 are close, because chip 2 has a pair of symmetry composite micropost arrays, the sorting throughput of chip 2 is two times that of chip 1 at the same flowing speed.

TABLE 4
Enrichment rates of tumor cells sorted by chip 2

| The ratio of HepG2 | The ratio of HepG2 cells | Enrichment rate |
|---|---|---|
| cells and blood | and blood cells(small | of tumor cells |
| cells(erythrocytes | amounts of residual | before and |
| and leukocytes) | leukocytes) | after |
| before sorting | after sorting | sorting |
| 124:1.1*10⁹ | 124:3.76*10⁴ | 2.92*10⁴ |

### Embodiment 4 Using a Composite Micropost Laterally-Displaced Chip to Sort Blood Cells

The purity of tumor cells of enrichment liquid and throughput are low when use the composite micropost laterally-displaced chips in embodiments 2 and 3 to sort blood cells. In order to improve the processing throughput and the purity of tumor cells, inlets and outlets of a chip 3 shown in FIG. 11 is adopted in present embodiment. The chip 3 is composed of two components, the first component composed of one or more pairs of symmetrical micropost arrays is connected to an inlet 4, and miropost units inside the first component are the composite microcolumns as shown in FIG. 5. The first component is used for enriching big size cells (tumor cells and part of big size leukocytes) in the blood in the middle location of symmetrical micropost arrays. The effect of enrichment is to improve the processing throughput; enriched cell solutions and buffer solutions from buffer inlet 7 pass through the second component together, and the second component is composed of lateral displacement micropost arrays, micropost units inside it are the composite microcolumns as shown in FIG. 5. Cells in enrichment cell solutions are separated according to the difference of sizes in the second component, finally target tumor cells are collected in the target particles outlet 5, waster liquids are collected in the waste liquid outlet 6.

The structure of microcolumns inside a chip is a kind of composite microcolumn shown in FIG. 5 with a length and a width of 15-70 μm, a row gap of 20-70 μm, a micropost gap of 20-70 μm, and an array displacement angle of 2-12 degree; width of small channels inside composite microposts is 4-12 μm, length and width of small rectangle arrays inside composite microposts is 3-30 μm, and height of composite microcolumns is 20-100 μm.

To be specific, A composite microcolumn has a length and a width of 50 μm, a row gap of 50 μm, a micropost gap of 50 μm, an array displacement angle in bottom component of 3 degree, an array displacement angle in top component of 3-6 degree which increasing progressively from left to right; width of small channels inside a composite micropost is 10 μm, length and width of a small rectangle micropost inside a composite micropost are 10 μm, and height of a composite microcolumn is 50 μm.

Passing a PBS buffer solution (pH 7.2 to 7.4, NaCl 137 mmol/L, KCl 2.7 mmol/L, Na₂HPO₄ 10 mmol/L, KH₂PO₄ 2 mmol/L) and a blood sample containing tumor cells respectively through two inlets of above chip, that the blood sample containing tumor cells flows through inlet 3 and the PBS buffer solution flows through inlet 4 at 3-5 mm/s. After sorting in the bottom component, an enriched blood solution (containing tumor cells and part of big size leukocytes) and a buffer solution enter into the top component, then different sizes cells are sorted, while tumor cells and big size leukocytes flow in a direction of lateral displacement of micropost arrays, erythrocytes and small size leukocytes flow along a direction of liquid, finally a target particles (tumor cells) outlet 5 and a waste liquid outlet 6 are used respectively to collect the tumor cells enrichment liquid and the waste liquid after sorting.

A chip 3 and above method are adopted to sort hepatoma carcinoma cells HepG2, and the concentration of tumor cells in a model sample about 187 cancer cells per milliliter. Table 4 gives a result of concentrations of tumor cells before and after sorting, showing that most of blood cells are filtrated, and tumor cells are enriched at an enrichment rate of 6.6×10⁵ after sorting. The enrichment rates of tumor cells sorted by chip 3 is one order higher than that of chip land chip 2, meanwhile, the throughput of chip 3 is well above that of chip land chip 2.

TABLE 5
Enrichment rate of tumor cells sorted by chip3

| The ratio of HepG2 | The ratio of HepG2 | |
|---|---|---|
| cells and blood | cells and blood | Enrichment |
| cells(erythrocytes | cells(small amounts | rate of tumor |
| and leukocytes) | of residual leukocytes) | cells before and |
| before sorting | after sorting | after sorting |
| 187:1.13*10⁹ | 187:1700 | 6.6*10⁵ |

### INDUSTRIAL APPLICATIONS

The present disclosure has the following advantages:

Each micropost unit inside a lateral displacement micropost array chips provided by the present disclosure has one or more small channels to form a new composite micropost, which can be used for sorting fluid and filtrating small particles (particles smaller than the width of channels); compared with a single microcolumn of continual cross section, the composite microcolumn can reduce the critical sorting size of micropost array under the same condition of microcolumn size and micropost lateral displacement angle; this kind of composite micropost array also provides another advantage that, compared with microcolumn of continual cross section, and under the same micropost size, a same critical sorting size can be obtained by adopting a larger array displacement angle. A larger array displacement angle produces a higher throughput to sort a bigger volume of sample, improving sorting efficiency.

1. A laterally-displaced micropost array chip, wherein each micropost unit is provided with one or more channels, and a channel is provided with at least one longitudinal runner and one transverse runner.

2. The laterally-displaced micropost array chip according to claim 1, wherein in one or more channels, one opening direction of at least one channel is different from the displacement direction of lateral displacement micropost arrays.

3. The laterally-displaced micropost array chip according to claim 1, wherein each micropost unit of a chip is composed of two or more independent microposts; and gaps between microposts form channels.

4. The laterally-displaced micropost array chip according to claim 1, wherein the chip is made of one or more of glass, silicon and a polymer; and the polymer is at least one of polymethyl methacrylate, bisphenol A polycarbonate, polycarbonates of 2,2-bis(4-hydroxyphenyl)propane, polystyrene, polyethylene, silicon resin, polyvinyl acetate, polypropylene, polyvinyl chloride, polyether ether ketone, polyethylene glycol terephthalate, cycloolefin polymer and cycloolefin copolymer. The cycloolefin of cycloolefin polymer and cycloolefin copolymer is selected from one or more of cyclopropene, cyclobutene, cyclopentene, cyclohexene, 1,3-Cyclobutadiene, cyclopentadiene and cyclohexadiene.

5. The laterally-displaced micropost array chip according to claim 4, wherein the chip comprises a substrate and/or a cover hermetically matched with the substrate;
the substrate or the cover is provided with lateral displacement micropost arrays;
one end of the chip is provided with a sample inlet to serve as a liquid sample inlet and/or buffer solution inlet, and the other end of the chip is provided with a waste liquid outlet used for collecting particles smaller than the critical sorting size and a target particles outlet used for collecting enriched particles greater than the critical sorting size.

6. A method for sorting a liquid sample consisting of various size particles using the laterally-displaced micropost array chip according to claim 1, comprising following steps: flowing the liquid sample consisting of various size particles through lateral displacement micropost arrays;
particles greater than the critical sorting size flowing along the displacement angle direction of the lateral displacement arrays to be collected in a target particles outlet;
particles both smaller than the critical sorting size and the channel smallest size keeping an original direction to be collected in a waste liquid outlet;
particles of various sizes are spatially separated.

7. The method for sorting a liquid sample consisting of various size particles according to claim 6, wherein the liquid sample comprises any one of:
(1) a Circulating tumor cell in a peripheral blood sample;
(2) a tumor cell in a pleural effusion, peritoneal effusion, lymph fluid, urine or bone marrow sample;
(3) a nucleated erythrocyte in a peripheral blood or umbilical cord blood sample;
(4) a circulating endothelial cell in the peripheral blood sample;
(5) a leukocyte, a T cell, a B cell, a lymphocyte, a monocyte, a granulocyte, a natural killer cell, a dendritic cell, a macrophage or a hematopoietic stem cell in a peripheral blood, umbilical cord blood, pleural effusion, peritoneal effusion, urine, cerebrospinal fluid or bone marrow sample;
(6) an erythrocyte or a platelet in a peripheral blood, umbilical cord blood, pleural effusion, peritoneal effusion, urine or bone marrow sample;
(7) a bacterium or a virus in a peripheral blood, pleural effusion, peritoneal effusion, urine, saliva, plasma, serum, cerebrospinal fluid, seminal fluid, prostatic fluid or vaginal secretion sample; and

8. (canceled)
